(19) United States
(12) Patent Application Publication
(10) Pub. No.: US 2011/0178210 A1
(43) Pub. Date: Jul. 21, 2011

(54) GELLED, FREEZE-DRIED CAPSULES OR AGGLOMERATES OF NANOOBJECTS OR NANOSTRUCTURES, NANOCOMPOSITE MATERIALS WITH POLYMER MATRIX COMPRISING THEM, AND METHODS FOR PREPARATION THEREOF

(76) Inventor: Pascal Tiquet, Grenoble (FR)

(21) Appl. No.: 13/056,600

(22) PCT Filed: Jul. 30, 2009
(86) PCT No.: PCT/EP2009/059901

§ 371 Date: Jan. 28, 2011

(30) Foreign Application Priority Data
Jul. 31, 2008 (FR) 0855311

(51) Int. Cl. C08L 5/00; C12P 19/04; C09K 3/00; B82Y 40/00

(52) U.S. Cl. 524/27; 435/101; 252/182.12; 977/898; 977/700; 977/773; 977/762; 977/788; 977/750; 977/752

(57) ABSTRACT

An agglomerate or capsule capable of being prepared by freeze-drying a first agglomerate or capsule, said first agglomerate comprising a solvent, nanoobjects or nanostructures coated with macromolecules of polysaccharides being homogeneously distributed in said agglomerate or said capsule, and said macromolecules forming in at least one portion of the first agglomerate, a gel by crosslinking with positive ions.

A nanocomposite material comprising this agglomerate.

A method for preparing this agglomerate and this nanocomposite material.

### TECHNICAL FIELD

The present invention is related to gelled, freeze-dried capsules or agglomerates of nanoobjects such as carbon nanotubes, or of nanostructures.

The present invention further relates to nanocomposite materials with a polymer matrix comprising these gelled, freeze-dried capsules or agglomerates, or prepared from these gelled, freeze-dried capsules or agglomerates.

The invention also relates to a method for preparing and conditioning these gelled, freeze-dried capsules or agglomerates, as well as to a method for preparing these nanocomposite materials with a polymer matrix from these gelled, freeze-dried capsules or agglomerates.

Finally, the invention relates to the uses of these gelled, freeze-dried agglomerates or capsules of nanoobjects.

### STATE OF THE PRIOR ART

The technical field of the invention may generally be considered as that of the inclusion, incorporation, confinement, containment, for various purposes, of nanoobjects such as nanoparticles in materials, such as polymers.

Thus, according to a first aspect of the invention, the technical field of the invention may more specifically be defined as that of the protection, confinement, containment of nanoparticles and nanoobjects with view to their handling.

It will also be noted that the invisible nature of these nanoobjects due to their small size and the lack of knowledge on their impacts on the biological world and living world also requires confinement, containment, and encapsulation for controlling dissemination and meeting the precautionary principle.

The technical field of the invention may, more specifically according to another aspect, be defined like that of composite materials, more specifically nanocomposite materials and notably nanocomposite materials with a polymer matrix.

The nanocomposite materials with a polymer matrix are multiphasic materials, in particular biphasic materials, which include a polymer matrix forming a first phase in which nanoobjects such as nanoparticles are dispersed, said nanoobjects forming at least one second phase which is generally called a strengthening or filler phase.

The nanocomposites are called in this way since at least one of the dimensions of the objects such as particles, forming the strengthening or filler phase is at a nanometric scale, i.e. generally less than or equal to 100 nm, for example of the order of one nanometer to one or a few tens of nanometers.

Accordingly, these objects and particles are called nanoobjects or nanoparticles.

With nanocomposites having a polymer matrix, for relatively low filler levels, i.e. less than 10% by weight, and even less than 1% by weight, a significant improvement of the properties of the material should theoretically be obtained, whether these are mechanical, electrical, thermal, magnetic or other properties . . . .

However, it has proved difficult to disperse nanoobjects homogeneously, notably at low concentrations, for example less than 1% by weight in polymer matrices. This difficulty of homogeneously dispersing nanoobjects, notably at low concentrations in polymer matrices, seems to come notably from the fact that these nanoobjects may appear as tubes, wires, nanolayers, or be bound to branched nanostructures and that these nanoobjects are entangled, aggregated and sometimes even branched.

Accordingly, notably at low concentrations of nanoobjects, improvements as to the properties of the materials which might be expected are not observed.

In other words, adding nanoobjects at low concentrations to polymers proves to be inefficient for actually improving the properties of these polymers because of the impossibility of obtaining a truly homogeneous dispersion of these nanoobjects in the polymer matrix.

In this respect, the example of carbon nanotubes (CNTs) is the most significant, since they have to be loaded with more than 5% by weight of the polymer matrix in order to expect to obtain a maximum improvement in the properties of the material, while theory predicts a percolation threshold at only 0.1% by weight of carbon nanotubes in the polymer [1].

In order to improve the dispersion of the nanoobjects in the polymer matrix and the compatibility of the nanoobjects with this polymer matrix, various techniques have successively been tested such as for example, chemical treatments and functionalizations, the use of surfactants and compatibilizing agents, and even polymerisation at the surface of nanotubes or nanostructures of thermoplastic polymers such as polyethylene (PE), polypropylene (PP), polystyrene (PS), or ethylene-norbornene copolymers. Thus, document [2] describes a nanocomposite with a polymer matrix comprising carbon nanotubes coated with a coating polymer, which is not miscible with the polymer of the polymer matrix.

For the polymers of the matrix with a polar nature such as polyamides, the techniques mentioned above may prove to be efficient and sufficient, but for aliphatic and apolar polymers such a polyethylenes (PE), polypropylenes (PP), either crosslinked or not, polystyrenes (PS), copolymers of cycloolefins (COC), with these techniques it is not possible to obtain a homogeneous dispersion of the nanoobjects in the matrix, and nor as a consequence, an improvement in the properties, notably at low concentrations.

The main reason of the insufficient efficiency of these techniques for accounting for compatibility (“compatibilization”) and improving homogeneity of the dispersion of the nanoobjects in the polymer matrix is that the nanoobjects, nanotubes or nanostructures of same nature are, at the end of their synthesis, totally entangled.

The use of powerful ultrasonic waves and/or powerful extruders does not give the possibility of finding a remedy to these problems since the thereby provided energy destroys the nanotubes or nanostructures without dispersing them, and further agglomerates them again.

The solution to the problem of accounting for compatibility (“compatibilization”) and improving the homogeneity of the dispersion of nanoobjects in a polymer matrix therefore neither lies in the functionalization of the nanoobjects, nor in the application of significant mechanical energy.

Besides, it is known that when the nanoobjects such as nanotubes, or the nanostructures are found in a liquid medium, in a diluted condition, at a low concentration for example less than or equal to 1% by mass, they are generally properly dispersed, i.e. dispersed in a homogeneous and organized way, and it would therefore be desirable to retain this organization.

However, up to now, it has not been possible to retain the same organization and the same homogeneity of the nanoobjects dispersed in a liquid medium in a composite material with a solid polymer matrix enclosing these nanoobjects and prepared from these dispersions.

Therefore, considering the foregoing, there exists a need for nanocomposite materials with a matrix polymer in which nanoobjects or nanostructures are dispersed, distributed, organized homogeneously, notably at a low concentration.

There further exists a need for a method with which such composite materials with a polymer matrix may be prepared, this method being further simple, reliable, reproducible and of low cost.

Further, there notably exists a need for materials ensuring confinement of nanoobjects with the purpose of controlling their dissemination in nature.

### SUMMARY OF THE INVENTION

The goal of the present invention is inter alia to meet these needs.

The goal of the present invention is notably to provide a nanocomposite material with a polymer matrix which does not have the drawbacks, defects, limitations and disadvantages of the nanocomposite materials of the prior art, and which solves the problems of the materials of the prior art.

The goal of the present invention is further to provide a method for preparing such a composite material with a polymer matrix which also does not have the drawbacks, defects, limitations and disadvantages of the methods for preparing nanocomposite materials of the prior art, and which solves the problems of the methods of the prior art.

The goal of the present invention in other words is to arrange that the organization and the homogeneity shown by the dispersed nanoobjects in a liquid medium are retained in a composite material with a solid polymer matrix prepared from these dispersed nanoobjects.

This goal and further other goals are achieved according to a first aspect of the invention by providing an agglomerate or a capsule capable of being prepared by freeze-drying of a first agglomerate or capsule, said first agglomerate or capsule comprising a solvent, nanoobjects or nanostructures coated with macromolecules of polysaccharides being distributed in a homogeneous way in said first agglomerate or capsule, and said macromolecules forming in at least one portion of the first agglomerate, a gel by crosslinking with positive ions.

The first agglomerate may be called for the sake of simplification, <<gelled agglomerate>> or <<gelled capsule>>.

The agglomerate prepared by freeze-drying of this first gelled agglomerate may be called for the sake of simplification <<freeze-dried gelled agglomerate>> or <<freeze-dried agglomerate>>.

By <<distributed in a homogeneous way>> is generally meant that the nanoobjects are uniformly distributed, regularly in the whole space of the first agglomerate and that their concentration is substantially the same in the whole space of the first agglomerate, in all the portions of the latter.

This homogeneous distribution is further retained in the freeze-dried agglomerate prepared from this first agglomerate.

The term of <<freeze-drying>> is a term well-known to the man skilled in the art. Freeze-drying generally comprises a freezing step during which the (liquid) solvent of the first agglomerate is put into solid form, for example as ice, and then a sublimation step during which, under the effect of a vacuum, the solid solvent such as ice is directly transformed into a vapor, for example steam, which is recovered. Possibly, once the whole (all the) liquid solvent, for example the whole of the ice, is removed, the agglomerates are dried under cold conditions.

The gel may be formed in the totality of the first agglomerate, or else the gel may be only formed in a portion of the first agglomerate, for example at the surface of the first agglomerate, the inside of the first agglomerate being in the liquid state.

Advantageously, the concentration of the nanoobjects or nanostructures (which is greater than 0% by mass) is less than or equal to 5% by mass, preferably it is less than or equal to 1% by mass, still preferably it is from 10 ppm to 0.1% by mass of the total mass of the first agglomerate.

The solvent of the first agglomerate may comprise in volume 50% of water or more, preferably 70% of water or more, still preferably 99% of water or more, better 100% water (the solvent of the first agglomerate is therefore then composed of water).

The solvent of the first agglomerate, when is does not comprise 100% of water, may further comprise at least one other solvent compound generally selected from alcohols, in particular aliphatic alcohols such as ethanol; polar solvents, in particular ketones, such as acetone; and mixtures thereof.

The solvent of the first agglomerate may further comprise a polymer soluble in said solvent.

The nanoobjects may be selected from nanotubes, nanowires, nanoparticles, nanocrystals and mixtures thereof.

The nanoobjects or nanostructures may be functionalized, notably chemically, in particular at the surface so as to introduce new functions via surface chemistry.

The material forming, constituting, the nanoobjects or nanostructures may be selected from carbon, metals, metal alloys, metal oxides such as optionally doped rare earth oxides, organic polymers, and materials comprising several of them.

Advantageously, the nanoobjects are carbon nanotubes (“CNT”), for example single-walled carbon nanotubes (<<SWCNT>>) or multi-walled carbon nanotubes (<<MWCNT>>), or nanoparticles of metals or metal alloys or metal oxides.

The polysaccharide macromolecules may be selected from pectins, alginates, alginic acid and carrageenans.

The alginates may be alginates extracted from brown algae Phaeophyceae, mainly Laminaria such as Laminaria hyperborea; and Macrocystis such as Macrocystis pyrifera.

Advantageously, the polysaccharide macromolecule has a molecular mass from 80,000 g/mol to 500,000 g/mol, preferably from 80,000 g/mol to 450,000 g/mol.

The first agglomerate or gelled agglomerate, notably in the case when it does not already further comprise a polymer soluble in the solvent of the first agglomerate, may be impregnated with at least one polymer or monomer soluble in the solvent of the first agglomerate, preferably with a water-soluble polymer selected for example from polyethylene glycols (PEG), poly(ethylene oxide)s, polyacrylamides, polyvinylpyridines, (meth)acrylic polymers, chitosans, celluloses, PVAs and all the other water-soluble polymers.

During freeze-drying, the solvent of the first agglomerate will be totally removed, replaced with the preferably water-soluble polymer or monomer, such as PEG impregnating the gelled agglomerate.

Also, during freeze-drying, the solvent of the first agglomerate or gelled agglomerate may be totally removed and replaced by the polymer or monomer soluble in the solvent of the agglomerate and already present in the agglomerate.

The first agglomerate may further be crosslinked and/or polymerized.

The freeze-dried agglomerate according to the invention generally contains from 1% to 90% by mass, preferably from 30% to 75% by mass, still preferably from 50% to 60% by mass, of nanoobjects or nanostructures, and from 10% to 99% by mass, preferably from 25% to 70% by mass, still preferably from 40% to 50% by mass of polysaccharide(s).

Advantageously, the freeze-dried agglomerate according to the invention may further after freeze-drying have undergone a heat treatment or an enzymatic treatment, attack.

This enzymatic attack may for example be achieved with an enzyme for degrading alginates, such as an enzyme of the Alginate Lyase type, such as the enzyme EC 4.2.2.3, also called E-poly(β-D-mannuronate) lyase.

The heat treatment or the enzymatic treatment gives the possibility of removing at least partly i.e. partly or completely, the polysaccharide of the agglomerate having undergone freeze-drying.

Generally, with the heat treatment, it is possible to remove at least partly the polysaccharide while with the enzymatic treatment, it is generally possible to totally remove the polysaccharide.

The enzymatic attack may be achieved according to standard conditions within the reach of the man skilled in the art, for example by putting the freeze-dried agglomerates into an aqueous solution and introducing the enzyme into the solution.

After this thermal or enzymatic treatment, the freeze-dried agglomerate generally contains from 50% to 100% by mass, preferably from 80% to 100% by mass of nanoobjects or nanostructures.

This heat or enzymatic treatment therefore gives the possibility of increasing the content of nanoobjects or nanostructures such as carbon nanotubes without changing the structure of the agglomerates, capsules and without affecting the homogeneous distribution of the nanoobjects or nanostructures in the agglomerate.

The additional heat treatment step which may also be called a step for calcination of the freeze-dried capsules, agglomerates or the additional enzymatic treatment step actually gives the possibility of at least partly removing the polysaccharide, for example the alginate, while retaining the organization previously obtained and notably the homogeneous distribution of the nanoobjects present in the first (gelled) agglomerates and in the freeze-dried agglomerates.

The additional heat treatment or enzymatic treatment step, carried out after freeze-drying therefore allows creation of agglomerates or capsules loaded with nanoobjects or nanostructures such that CNTs with a very high content which may notably range from 80% to 95% by mass of the agglomerate.

Such a high content is obtained even with a very low content of nanoobjects or nanostructures such as CNTs in the gelled agglomerates, since the tubes for example are generally long with a length for example comprised between 1 μm and 100 μm.

Such a content is greater than all the contents of nanoobjects or nanostructures obtained hitherto in such agglomerates or capsules and this without affecting the homogeneous distribution of these nanoobjects or nanostructures, their three-dimensional organization, already present both in the first agglomerates and in the freeze-dried agglomerates, in the agglomerates after a heat treatment which may also be called <<calcinated>> agglomerates or in the agglomerates after enzymatic treatment.

In other words, the heat treatment step or calcination step, or the enzymatic treatment step, aims at totally or partially removing the polysaccharide in the freeze-dried agglomerate. At the end of the heat, treatment step, calcinations step, or enzymatic treatment step, carried out after freeze-drying, structures are obtained which may be exclusively formed with nanoobjects or nanostructures (when the polysaccharide such as the alginate has been totally removed) such as CNTs, these structures being organized and porous, which is an advantage for integrating these structures into certain polymers.

The polysaccharide content in the agglomerates after heat or enzymatic treatment is generally from 1% to 50% by mass, preferably from 1% to 20% by mass, or even 0% by mass, notably when an enzymatic treatment, attack is carried out.

The invention further relates to the use of the freeze-dried agglomerate as described above (also optionally having a heat or enzymatic treatment) in microfluidic systems, or as a metamaterial notably for simulating the behavior of plasmas under electromagnetic radiation.

The invention also relates to a nanocomposite material with a polymer or composite matrix comprising an agglomerate or a first gelled agglomerate as defined above (whatever it may be) in which the nanoobjects or nanostructures are distributed homogeneously.

The polymer(s) of the matrix may be selected from aliphatic and apolar polymers such as polyolefins, such as polyethylenes and polypropylenes, polystyrenes, copolymers of cycloolefins; but also from polar polymers such as polyamides and poly(meth)acrylates such as PMMA; and mixtures thereof.

The polymer of the matrix may also be selected from polymers which melt or which are soluble in water.

The composite of the matrix may be selected from composite materials comprising at least one polymer for example selected from the polymers mentioned above for the matrix, and an inorganic filler.

The invention further relates to a method for preparing the agglomerate as defined above, wherein the following successive steps are carried out:
a) Nanoobjects or nanostructures are dispersed in a first solvent comprising water in majority; polysaccharide macromolecules and optionally a polymer or monomer soluble in the first solvent are put into solution into the first solvent, whereupon a first solution is obtained;
b) a third solution is prepared by putting the first solution into contact with a second solution in a second solvent comprising water in majority, of at least one salt soluble in water, capable of releasing into the second solution monovalent, divalent or trivalent cations, whereupon a first agglomerate is obtained;
c) the first agglomerate is separated from the third solution;
d) the first agglomerate is freeze-dried;
e) optionally, a heat or enzymatic treatment of the first freeze-dried agglomerate is carried out.

The first solvent may comprise in volume 50% of water or more, preferably 70% by volume of water or more, still preferably 99% by volume of water or more, and better 100% by volume of water.

The nanoobjects, nanostructures, and the polysaccharides are advantageously such as they have been already defined above.

The first solvent when it does not comprise 100% water, may further comprise at least one other solvent compound generally selected from alcohols, in particular aliphatic alcohols such as ethanol; polar solvent compounds in particular ketones such as acetone; and mixtures thereof.

The dispersion of the nanoobjects in the solvent and the putting of the polysaccharides into solution may be two simultaneous operations, or else they may be two consecutive operations, dispersion preceding the putting into solution, or vice versa.

Advantageously, the ratio of the number of macromolecules to the number of nanoobjects in the first solution may be from 1 to 10, preferably this ratio is equal to or close to 1.

The content of nanoobjects and the content of macromolecules of polysaccharides (which are more than 0% by mass) may advantageously be less than or equal to 5% by mass, preferably less than or equal to 1% by mass, and still preferably from 10 ppm to 0.1% by mass of the mass of the first solvent.

The second solvent may comprise 50% by volume of water or more, preferably 70% by volume of water or more, still preferably 99% by volume of water or more, better 100% by volume of water.

The second solvent may further comprise, when it does not comprise 100% water, at least one other solvent compound generally selected from alcohols, in particular aliphatic alcohols such as ethanol; polar solvents in particular ketones such as acetone; and mixtures thereof.

Advantageously, the second solvent is identical with the first solvent.

Advantageously, the bivalent cations may be selected from Cd²⁺, Cu²⁺, Ca²⁺, Co²⁺, Mn²⁺, Fe²⁺, Hg²⁺; the monovalent cations may be selected from Li⁺, Na⁺, K⁺, Rb⁺, Cs⁺, Ag⁺, Ti⁺, Au⁺; and the trivalent cations may be selected from Fe³⁺ and Al³⁺. The preferred cations are Cu²⁺, Ca²⁺ or Fe³⁺.

Advantageously, the second solution may comprise several salts so that a mixture of cations, preferably a mixture of cations comprising at least one monovalent cation, at least one divalent cation, and at least one trivalent cation may be released in the second solution.

The method for preparing the first agglomerate is reversible and may optionally further comprise a step c1) (carried out on the agglomerate obtained at the end of step c)) during which the first agglomerate is put into contact with at least one chelating agent such as diethylene tetramine pentaacetic acid (DTPA), ethylene diamine tetraacetic acid, or trientine (triethylene tetramine, TETA) for scavenging the cations and deactivating the role thereof.

At the end of step b) or of step c), the agglomerate having been obtained, and optionally separated, for example by simple filtration, may be impregnated with a solution of a polymer or monomer soluble in the first solvent, preferably with an aqueous solution of at least one water-soluble polymer or monomer for example selected from polyethylene glycols (PEG), poly(ethylene oxide)s, polyacrylamides, polyvinyl pyridines, (meth)acrylic polymers, chitosans, celluloses, PVAs and all the other water-soluble polymers, and it is then proceeded with freeze-drying of the first agglomerate impregnated according to step d).

However, as this has already been mentioned, a polymer or monomer may also be added during step a) so as to mechanically consolidate the solution of nanoobjects dispersed by means of the polysaccharide, said polymer or monomer then being soluble in the solvent (<<first solvent>>) used in step a). This may be in particular a water-soluble monomer or polymer which may be selected from the polymers already mentioned above.

The freeze-drying step may be carried out on the first agglomerate whether it comprises a polymer or monomer added during step a) or not and whether it has been impregnated or not with a solution of a polymer or monomer, for example with an aqueous solution of a water-soluble polymer or monomer at the end of step b) or of step c).

During step e), a heat or enzymatic treatment is optionally carried out on the freeze-dried agglomerate.

The heat or enzymatic treatment has the purpose of removing at least partly the polysaccharide still present.

Generally, at least 30% by mass of the polysaccharide present in the freeze-dried agglomerates, for example from 30% to 45% by mass, is removed by this heat treatment. It is even possible to totally remove the polysaccharide with the enzymatic attack.

At the end of this heat or enzymatic treatment an agglomerate is obtained generally comprising from 0% to 50% by mass, preferably from 0% to 20% by mass of polysaccharide, and from 50% to 100% by mass, and preferably from 80% to 100% by mass of nanoobjects or nanostructures.

The heat treatment should be carried out at a temperature such that it allows at least partial removal of the polysaccharide from the freeze-dried agglomerates.

Advantageously, it is carried out at a temperature from 400° C. to 600° C., preferably from 500° C. to 550° C., for a duration from one to five hours, preferably from one to three hours, still preferably from one to two hours, for example at a temperature of 300° C. for one hour.

The enzymatic treatment conditions as this has already been indicated above may be easily determined by one skilled in the art.

The invention finally relates to a method for preparing a nanocomposite material in which it is proceeded with the incorporation of at least one (freeze-dried) agglomerate possibly thermally or enzymatically treated or of at least one first agglomerate as defined in the foregoing in a polymer or composite matrix.

In other words, it is possible to incorporate into the polymer or composite matrix, a gelled agglomerate, a freeze-dried agglomerate or a heat-treated agglomerate, calcinated agglomerate or an enzymatically treated agglomerate.

The polymer of the matrix has already been defined above.

The incorporation of the (freeze-dried and optionally thermally or enzymatically treated), agglomerate or of the first agglomerate into the polymer matrix may be carried out by a plastic engineering, processing, method such as extrusion.

Extrusion consists of melting n-materials and of kneading them along a screw or a twin screw with optimized temperature profile, pattern, and speed of rotation in order to obtain an optimum mixture.

At the end of the twin screw or single screw, a die is found which shapes the mixture before its complete solidification. The shape may be a string or cord, a film, or may have any type of profile.

The agglomerates according to the invention, as such, have never been described nor suggested in the prior art, they give the possibility for the first time of retaining in the final solid nanocomposite material according to the invention the same organization, notably the same homogeneous distribution of the nanoobjects or nanostructures, as the one which existed in the dispersion of these nanoobjects or nanostructures in a liquid medium.

According to the invention, this organization is retained in the first agglomerate, and then in the freeze-dried agglomerate and then in the agglomerate having undergone the heat or enzymatic treatment.

In fact, the gelled structure of the agglomerates according to the invention gives the possibility of setting, fixing, <<freezing>> in a stable way the organization of the nanoobjects or nanostructures, for example the homogeneous distribution, which was the one of the nanoobjects in the liquid dispersion, and of subsequently retaining it entirely in the final composite material.

The invention provides a solution to the problems of the prior art and meets the whole of the needs listed above.

In particular, by means of the agglomerates of the invention, it is unexpectedly possible to retain the state of dispersion of the nanoobjects or nanostructures which exists in the initial dispersion in the final nanocomposite material which may then be treated, converted in a conventional way by any plastic engineering, processing, method, for example by extrusion.

This fundamental problem was never able to be solved in the prior art and formed what is customarily called a <<technological bottle-neck>> or “technological lock” which may be removed by the invention.

In the final composite material, the same homogenous distribution of the nanoobjects or nanostructures as in the initial dispersion is therefore found again in the whole of the volume of the material.

The nanocomposite materials according to the invention are neither described nor suggested in the prior art and intrinsically differ from the nanocomposite materials of the prior art, notably by the fact that they comprise the first agglomerates or the agglomerates according to the invention, which impart intrinsically novel and unexpected properties to them as compared with the nanocomposite materials of the prior art, in particular as regards the homogeneity of the distribution of the nanoobjects or nanostructures at low contents, concentrations.

Indeed, this preservation of the state, which was that of the nanoobjects or nanostructures in the initial dispersion, also in the final composite material is intimately related to the application, use, of the particular <<gelled>> agglomerates according to the invention, and is in particular observed surprisingly for a low concentration of nanoobjects or nanostructures, i.e. a concentration generally less than or equal to 5% by mass, preferably less than or equal to 1% by mass, preferably from 10 ppm to 0.1% by mass in the composite material.

But the invention may also be applied, carried out, advantageously for high concentrations of nanoobjects or nanostructures, for example a concentration which may range up to and close to 20% by mass. At these high concentrations, the method according to the invention gives the possibility of controlling the organization, the arrangement and the level of entanglement.

Generally, the concentration of nanoobjects or nanostructures will therefore be from 10 ppm to 20% by mass, preferably from 10 ppm to 5% by mass, still preferably from 10 ppm to 1% by mass and better from 10 ppm to 0.1% by mass in the final composite material.

The problem of the homogeneous dispersion of nanoobjects or nanostructures at low concentrations in nanocomposites was posed with particular acuteness and had never received any solution, at least any satisfactory solution, in the prior art.

Because of the homogeneous distribution of the nanoobjects, nanostructures obtained according to the invention at a low level, at a low concentration, i.e. generally less than or equal to 5% by mass, preferably less than or equal to 1% by mass, an improvement in the (mechanical, electrical, thermal, magnetic . . . ) properties due to these nanoobjects such as carbon nanotubes, or nanostructures is observed at lower concentrations. Significant savings of materials that are often costly on the one hand, and for which the synthesis methods are not adapted to mass production, on the other hand, are thereby achieved.

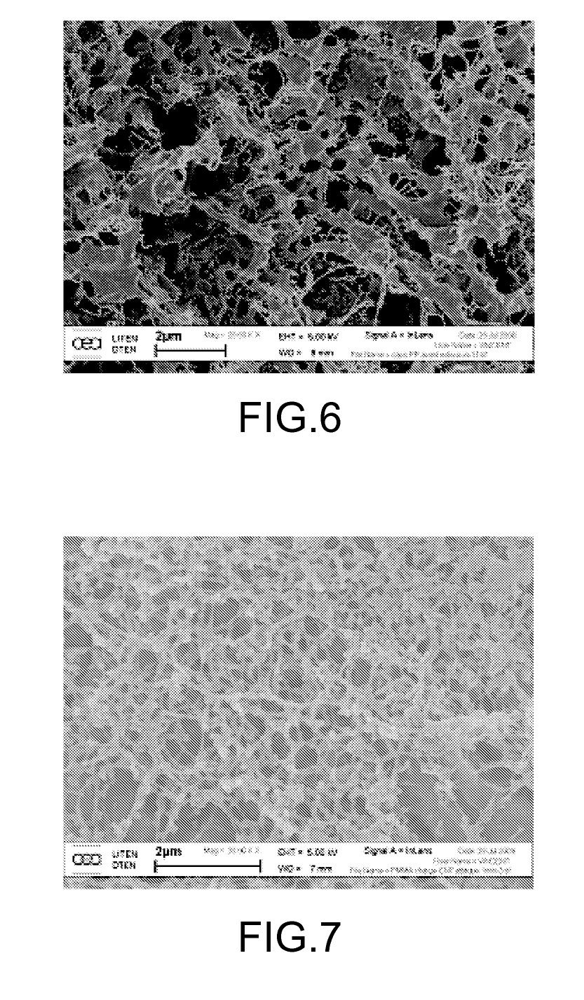

The shape, the properties of the nanoobjects are not affected in the agglomerates according to the invention and then in the composite materials according to the invention, they do not undergo any degradation both in the agglomerates and in the composite material (see FIGS. 6 and 7).

The invention will be better understood upon reading the detailed description which follows, made as an illustration and not as a limitation with reference to the appended drawings wherein:

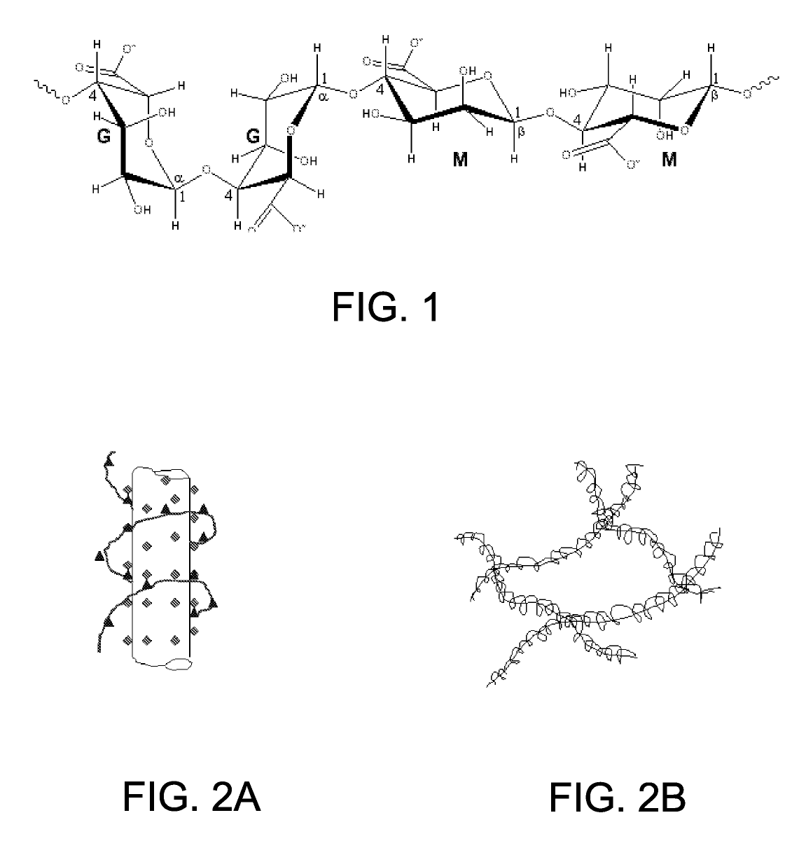

FIG. 1 shows the chemical structure of a polysaccharide molecule, which is an alginate stemming from brown algae Phaeophyceae;

FIG. 2A shows at a nanometric scale the winding of a polysaccharide macromolecule around a multi-wall carbon nanotube (MWCNT) by electrostatic interaction of the acid sites, the rhombs, losanges (♦) represent the acid sites of the carbon nanotubes, while the triangles (▴) represent the acid sites of the polysaccharide macromolecule;

FIG. 2B illustrates the nanostructure in a lattice of multi-wall carbon nanotubes (MWCNT) with the polysaccharide molecules, the carbon nanotubes are illustrated by solid lines and the polysaccharide macromolecules are illustrated by windings around these solid lines;

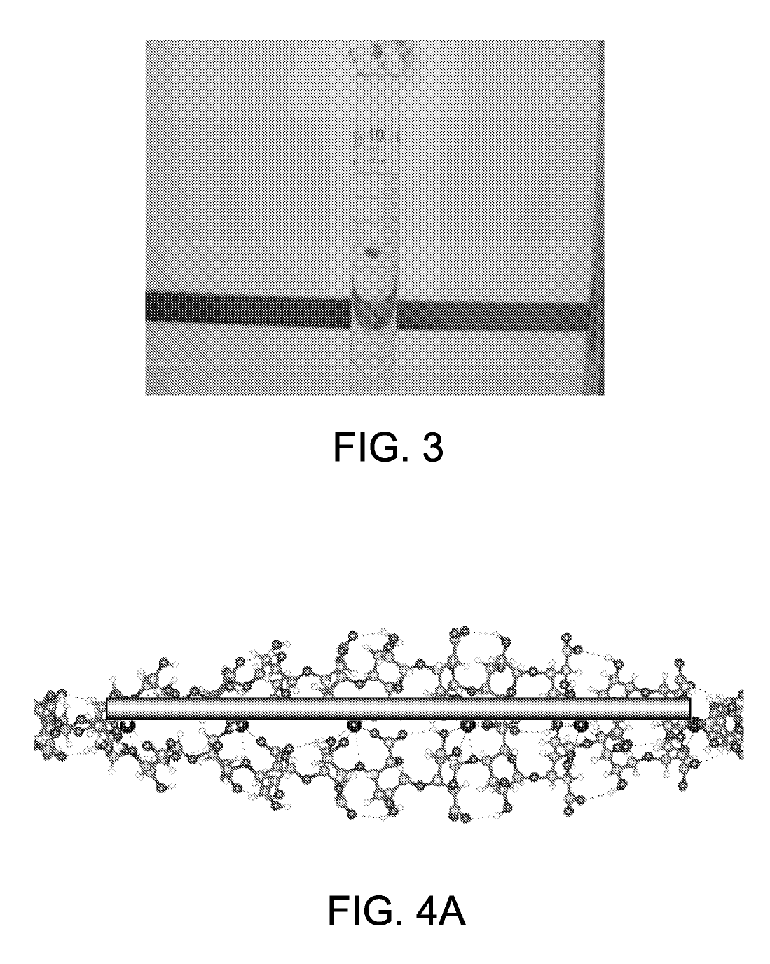

FIG. 3 is a photograph which shows an example of the formation of an agglomerate according to the invention in a test tube, with nanotubes as nanoobjects;

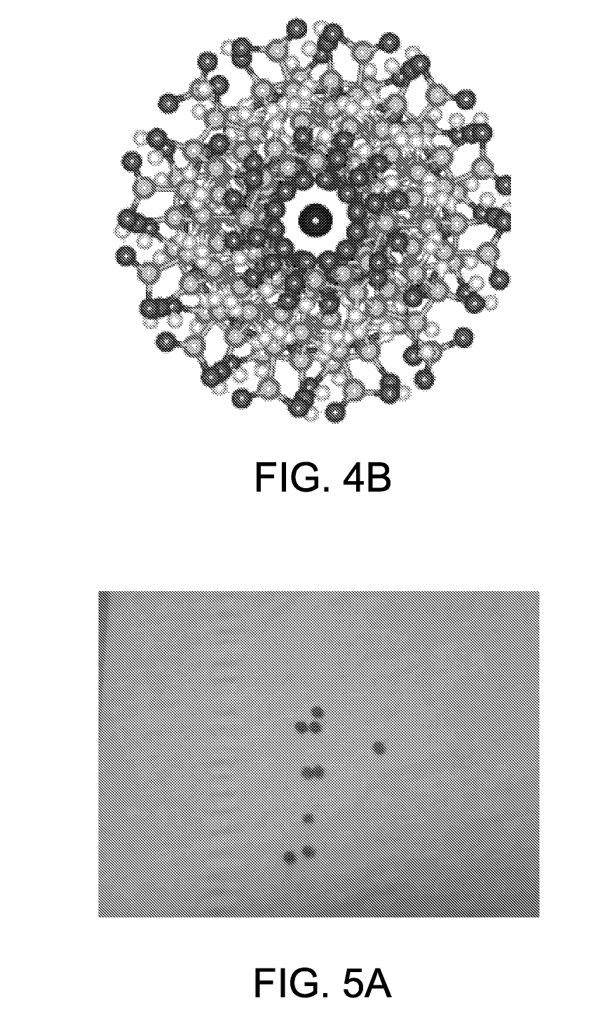

FIGS. 4A and 4B are respectively longitudinal and axial schematic views, showing an organization example at a nanometric scale of a gelled agglomerate according to the invention, a carbon nanotube is found in the center of this agglomerate;

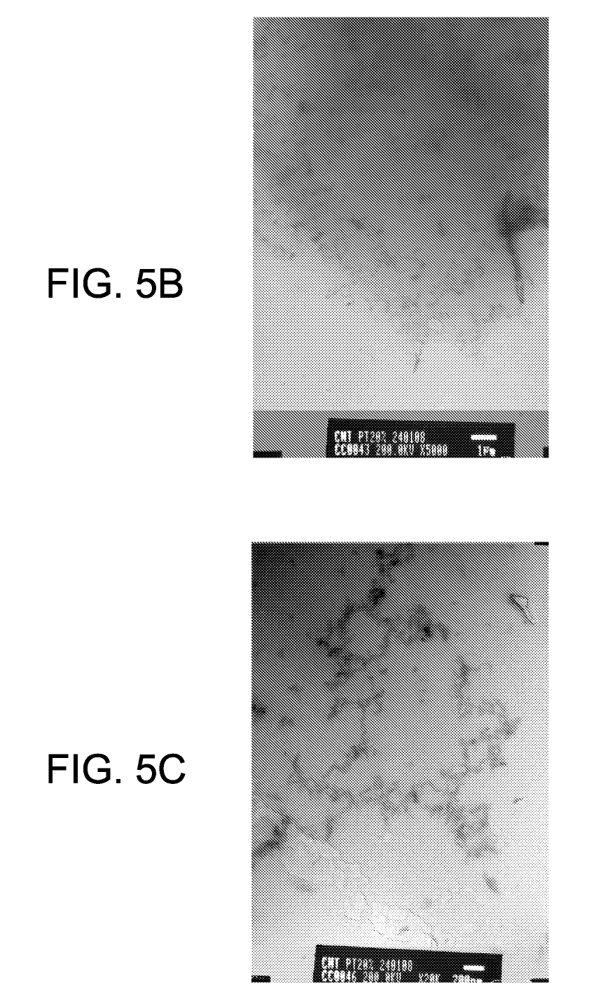

FIGS. 5A, 5B and C are photographs at a respectively millimetric, micrometric and nanometric scale showing the optimum dispersion of the lattice of nanotubes in a material according to the invention.

The scale illustrated in FIG. 5B is 1 μm, and the scale illustrated in FIG. 5C is 200 nm.

FIG. 6 is a photograph taken with a microscope, showing the organization of CNTs in a capsule according to the invention after freeze-drying;

The scale illustrated in the figure is 2 μm.

FIG. 7 is a photograph taken with a microscope, showing the organization of CNTs after mixing capsules according to the invention with PMMA (PolyMethyl MethAcrylate).

The scale illustrated in the figure is 2 μm.

The detailed description which follows, is rather made in connection with the method according to the invention for preparing <<gelled>> agglomerates, freeze-dried agglomerates and nanocomposite materials with a polymer matrix but it also includes teachings which apply to the agglomerates and to the materials according to the invention.

As a preamble to this detailed description, we first of all specify the definition of certain of the terms used herein.

By nanoobjects, are generally meant any object alone or connected, bound, to a nanostructure for which at least one dimension is less than or equal to 100 nm, for example of the order of one nanometer to one or a few tens of nanometers.

These nanoobjects may for example be nanoparticles, nanowires, nanotubes, for example single-walled carbon nanotubes (CNT) (SWNT or single-walled nanotubes).

By nanostructure, is generally meant an architecture consisting of an assembly of nanoobjects which are organized with a functional logic and which are structured in a space ranging from one cubic nanometer to one cubic micrometer.

By polysaccharide, is generally meant a polymeric organic macromolecule consisting of a chain of monosaccharide units. Such a macromolecule may be represented by a chemical formula of the form —[Cₓ(H₂O)_{y}]ₙ—.

By agglomerate (or capsule), is generally meant a system comprising, preferably consisting of, composed of a solvent, preferably a solvent comprising water in majority or consisting of water; nanoobjects or nanostructures; polysaccharide macromolecules; and positive ions playing the role of crosslinking nodes between two polysaccharide molecules.

The term of metamaterials in physics, in electromagnetism, generally designates on the whole artificial composite materials and nanocomposites which have electromagnetic properties which are not found in natural materials.

A definition of nanocomposite materials with a polymer matrix has already been given above.

The method according to the invention may be defined as a method for preparing <<gelled>>, freeze-dried and optionally calcinated or enzymatically treated agglomerates (or capsules) of nanoobjects or nanostructures.

In a first step, nanoobjects or nanostructures are dispersed in a first solvent generally comprising water in majority, and at least one macromolecule belonging to the family of polysaccharides is put into solution in the first solvent, as a result of which a first solution is obtained in which the nanoobjects or nanostructures are dispersed.

At this stage of the method, it is possible to add to the first solution a polymer or monomer soluble in the first solvent, for example water-soluble, the function of which will be to maintain the gel (gelled) structure when the first solvent, such as water, will have left.

By solvent comprising water in majority, it is generally meant that the solvent comprises 50% by volume or more of water, preferably 70% by volume or more of water, and still preferably more than 99% by volume of water, for example 100% water.

The first solvent may comprise in addition to water in the aforementioned proportions at least one other solvent compound, generally selected from alcohols, in particular aliphatic alcohols such as ethanol; polar solvents, in particular ketones such as acetone; and their mixtures.

In addition to the aforementioned solvents, the first solution may, as specified above, further contain at least one polymer selected from all the polymers soluble in the first solvent, notably water-soluble polymers such as PEGs, poly(ethylene oxide)s, polyacrylamides, polyvinyl pyridines, (meth)acrylic polymers, celluloses, chitosans, PVAs, having the function of efficiently stabilizing the dispersion of nanoobjects, nanostructures.

The nanoobjects are such as defined above and may be nanotubes, nanowires, nanoparticles, nanocrystals or a mixture thereof.

The material making up these nanoobjects or nanostructures is not particularly limited and may be selected from carbon, metals and metal alloys, metal oxides such as optionally doped rare earth oxides, organic polymers; and mixtures thereof.

Preferred nanoobjects are notably carbon nanotubes (CNTs) whether these are single-wall carbon nanotubes (SWCNTs) or multi-wall carbon nanotubes (MWCNTs), nanoparticles of metals or alloys, nanoparticles of <<tracers>> i.e. optionally doped rare earth oxides.

The nanostructures may be constructions, assemblies for which the bricks are nanoobjects.

The nanostructures may be for example carbon nanotubes “decorated” with platinum, copper, gold nanoparticles; silicon nanowires <<decorated>> with gold, nickel, platinum etc.

Among the nanostructures, mention may notably also be made of the nanostructure ZnO—Ni which is a three-dimensional structure of ZnO terminated by nickel nanospheres.

The agglomerates may only contain a single type of nanoobject or nanostructure but they may contain both (at the same time) several types of nanoobjects and/or nanostructures which may differ by their shape and/or the material making them up, constituting them, and/or their size.

For example, an agglomerate may contain both carbon nanotubes and metal nanoparticles such as copper.

There exists no limitation as to the polysaccharide macromolecule and all the molecules belonging to the family of polysaccharides may be used in the method according to the invention. These may be natural or synthetic polysaccharides.

The polysaccharide macromolecule may be selected from pectins, alginates, alginic acid and carageenans.

By alginates are meant both alginic acid and the salts and derivatives of the latter such as sodium alginate. The alginates and notably sodium alginate are extracted from various brown algae Phaeophyceae, mainly Laminaria such as Laminaria hyperborea; and Macrocystis such as Macrocystis pyrifera. Sodium alginate is the most current marketed form of alginic acid.

Alginic acid is a natural polymer of raw formula (C₆H₇NaO₆)ₙ consisting of two monosaccharide units; D-mannuronic acid (M) and L-guluronic acid (G) (FIG. 1). The number of base units of the alginates is generally about 200. The mannuronic acid and guluronic acid proportion varies from one algae species to another and the number of units (M) on the number of units (G) may range from 0.5 to 1.5, preferably from 1 to 1.5.

The alginates are linear non-branched polymers and are not generally random copolymers but depending on the algae from which they stem, they are formed with sequences of similar or alternating units, i.e. GGGGGGGG, MMMMMMMM, or GMGMGMGM sequences.

For example, the ratio M/G of the alginate stemming from Macrocystis pyrifera is about 1.6 while the ratio M/G of the alginate stemming from Laminaria hyperborea is about 0.45.

Among the polysaccharide alginates stemming from Laminaria hyperborea, mention may be made of Satialgine SG 500, among the polysaccharide alginates stemming from Macrocystis pyrifera with different molecule lengths, mention may be made of the polysaccharides designated as A7128, A2033 and A2158 which are generics of alginic acids.

The polysaccharide macromolecule applied, used, according to the invention generally has a molecular mass from 80,000 g/mol to 500,000 g/mol, preferably from 80,000 g/mol to 450,000 g/mol.

The dispersion of the nanoobjects or nanostructures in the first solvent and the putting into solution of the polysaccharides may be two simultaneous operations or else this may be two consecutive operations, the dispersion preceding the putting into solution or vice versa.

The dispersion of the nanoobjects such as nanotubes, or of the nanostructures in the first solvent may be accomplished by adding the nanoobjects to the first solvent and submitting the solvent to the action of ultrasound with an acoustic power density generally from 1 to 1000 W/cm², for example 90 W/cm², for a duration generally from five minutes to twenty-four hours, for example two hours.

The putting of the polysaccharides into solution may be accomplished by simply adding said polysaccharides to the first solvent under stirring generally at a temperature from 25° C. to 80° C., for example 50° C., for a duration generally from five minutes to twenty-four hours, for example two hours.

The nanoobjects or nanostructures content and the polysaccharides content depend on the amount of nanoobjects and of nanostructures to be coated as compared with the amount of polysaccharide molecules.

The content of nanoobjects in the first agglomerate, or gelled agglomerate, as well as the polysaccharide content are generally less than or equal to 5% by mass, preferably less than or equal to 1% by mass, of the mass of the solvent. It was seen above that the invention at such <<low>> concentrations gives the possibility of obtaining particularly advantageous effects. Still preferably, the content of nanoobjects and the content of polysaccharides are from 10 ppm to 5% by mass, still preferably from 10 ppm to 1% by mass, and better from 10 ppm to 0.1% by mass of the mass of the solvent in the first agglomerate or gelled agglomerate.

The ratio of the number, of the amount, of macromolecules to the number of nanoobjects in the first solution and consequently in the first agglomerates or gelled agglomerates, is generally from 0.1 to 10, preferably equal to or close to 1.

This ratio between the amount, the number of polysaccharide molecules and the amount, the number of nanoobjects or nanostructures sets the dispersion level or dispersion factor and the average distance for the nanoparticles, or sets the unit cell of the lattice for nanostructures, nanowires and nanotubes.

If multi-wall carbon nanotubes (MWCNT) are taken as an example of nanoobjects, the smallest one of these tubes measures on average 1.5 nm and the largest 20 nm.

A multi-wall nanotube contains on average nanotubes fitted into each other over an average length of 1 μm. A solution of 100 ml of water containing 0.1% of MWCNT leads to the dispersion of about 10¹⁶ nanoobjects in 100 ml of water. The minimum amount of 10¹⁶ polysaccharide macromolecules, for example corresponds for the polysaccharide of the algae Phaeophyceae to a minimum amount of 20% by mass, for a molar mass of polysaccharide such as an alginate comprised between 80,000 g/mol and 120,000 g/mol. With these optimum amounts of nanotubes and of polysaccharides, each polysaccharide macromolecule is helically wound around a nanotube in order to minimize the electrostatic interaction energies between the O⁻ of the polysaccharide molecule (FIG. 1) and the acid sites of the MWCNTs (FIG. 2A).

In FIG. 1, an exemplary chemical structure is given of a polysaccharide macromolecule, i.e. an alginate molecule stemming from the brown algae Phaeophyceae. It is well understood that any molecule belonging to the family of polysaccharides may be used in the method according to the invention and that the explanations given herein apply to any polysaccharide macromolecule. Also the present description applies to any nanoobject, to any nanostructure and is not limited to nanotubes.

The presence of —OH bonds and of anionic functions —O⁻ in the chemical structure of the polysaccharide as the one illustrated in FIG. 1 gives the possibility of respectively ensuring solubilization in the solvent, i.e. generally essentially in water, and encapsulation, coating of the nanoobjects such as nanotubes or of the nanostructures because of the electrostatic attraction between the polar functions and acid functions. The helical structure of the polysaccharides allows the winding of these macromolecules around the nanoobjects notably around the carbon nanotubes.

The topology of the macromolecule at a nanometric scale is illustrated in FIGS. 2A and 2B.

FIG. 2A shows the winding of a polysaccharide molecule like the one in FIG. 1 around a multi-walled carbon nanotube by electrostatic interaction of the acid sites, while FIG. 2B shows the nanostructure of a lattice of multi-walled carbon nanotubes with polysaccharide molecules such as those of FIG. 1.

As this has already been indicated above, the ratio of the amounts of polysaccharide macromolecules and the amount of nanoobjects or nanostructures such as carbon nanotubes, sets the size of the unit cell of the lattice of nanoobjects or nanostructures such as carbon nanotubes and therefore the dispersion factor.

For nanotubes with an average length of 1 μm, the size of the maximum unit cell for the percolation of the four faces of a cube of 1 μm³ is a unit cell of 1 μm×1 μm. At least three carbon nanotubes (CNTs) are required for percolating all the faces of the cube, which corresponds by a change in scale to an amount of 3.10¹² CNTs for 1 cm³ of solution and 3.10¹⁴ CNTs for 100 ml.

This concentration of CNTs corresponds to a mass ratio of 0.1% by weight.

For a mass ratio which is ten times larger, i.e. 1%, the size of the unit cell will be reduced by a factor of 10.

The optimum of the mixture will always be achieved when the polysaccharide/nanoobjects ratio (for example nanotubes) is close to 1. It is the concentration of the species which determines the size of the unit cell.

In a second step, gelled agglomerates (first agglomerates) are prepared such as those shown in FIG. 3 by putting the first solution of dispersed nanoobjects prepared during the first step, described above, into contact with a second solution. This second solution is a solution, in a second solvent comprising water in majority, of at least one water-soluble salt capable of releasing into the solution, cations selected from monovalent, divalent and trivalent cations.

By solvent comprising water in majority, is generally meant that the solvent of the second solution comprises 50% by volume or more of water, preferably 70% by volume or more of water, and still more preferably more than 99% by volume of water.

The solvent may comprise, in addition to water in the aforementioned proportions and when it does not comprise 100% water, at least one other solvent compound generally selected from alcohols, in particular aliphatic alcohols such as ethanol; polar solvents such as ketones for example acetone; and their mixtures.

The divalent cations may be selected from Cd²⁺, Cu²⁺, Ca²⁺, Co²⁺, Mn²⁺, Fe²⁺, and Hg²⁺.

The monovalent cations may be selected from Li⁺, Na⁺, K⁺, Rb⁺, Cs⁺, Ag⁺, Ti⁺, and Au⁺.

The trivalent cations may be selected from Fe³⁺, and Al³⁺.

The anion of the salt(s) may be selected from nitrate, sulphate, phosphate ions, halide ions such as chloride, bromide ions.

The solution may only comprise a single salt or else it may comprise several salts.

Advantageously, the solution comprises several salts so that a mixture of cations may be released into the second solution.

Preferably, the solution comprises a mixture of salts which may release in the solution a mixture of cations comprising at least one monovalent cation, at least one divalent cation, and at least one trivalent cation.

With a mixture of cations selected from the three families of monovalent, divalent and trivalent cations and preferably comprising at least one cation selected from each of the families, it is possible to control the amount of crosslinking nodes of the system, and it is notably possible to minimize this amount of crosslinking nodes in order to thereby ensure structural stability of the gelled agglomerates and then of the freeze-dried agglomerates.

Indeed, the amount of crosslinking nodes is a parameter which has to be controlled depending on the use which is made of the agglomerates and of their applications.

The putting of the first solution and of the second solution into contact is generally achieved under the following conditions:

In a first embodiment of this putting into contact, the solution of dispersed nanoobjects or nanostructures falls dropwise into the second solution. In this case, the size of the endpiece, tip, is important since it conditions the size of the gelled agglomerate. If this is too large, freeze-drying, extraction of water for example, takes place moderately well and shrinkage is more significant, therefore the dispersion is not as good.

If they are too small, the agglomerates freeze-dry perfectly, but the time for preparing these gelled agglomerates is incredibly long. The optimum of the size of the spray nozzle is comprised between 0.5 and 2 mm, ideally 1 mm.

According to the conditions of the putting into contact and to the nature of the nanoobjects or nanostructures, it is possible to make spherical gelled agglomerates or else filamentary and stretched gelled agglomerates with controlled drawing ratios.

In a second embodiment of this putting into contact and unlike the drop by drop (dropwise) technique, continuous contacting is achieved with the crosslinking solution with a spray nozzle directly placed in the crosslinking solution.

The shape and the size of the spray nozzle, and in particular the ratio of the diameter of the inlet cylinder on the diameter of the outlet cylinder and the length of the latter condition the drawing ratio of the nanoobjects such as carbon nanotubes.

As an example, an inlet and outlet diameter of 2 mm and 50 μm respectively gives a drawing ratio of 400%. By doubling the inlet diameter for a same outlet diameter, the drawing ratio is multiplied by four so as to reach 1600%.

This type of drawing, if need be, allows alignment of the nanoobjects such as carbon nanotubes. If this spray nozzle is equipped with electrodes for generating an electric field, this allows organization of the nanostructures just before gelling.

The spherical gelled agglomerates may have a size from 100 μm to 5 mm and the filamentary agglomerates may have a size from 10 μm to 5 mm.

Accordingly it is possible to control the orientation of the nanoobjects or nanostructures in the gelled agglomerate, which are aligned in the case of maximum drawing, or else which are oriented in a purely random way but distributed regularly, homogeneously in the case of spherical agglomerates.

It is also possible to only form a crosslinked skin and to retain the inside of the first agglomerates in the liquid state. This may be obtained by projecting with a <<spray>> the crosslinking solution on the liquid drop being formed before its detachment from the spray nozzle. It is thereby possible to retain great mobility of the nanoobjects inside the capsules.

In certain fields, high performance metamaterials are required, while retaining high electric or magnetic permittivity values at very high frequencies. The mobility of the charge carriers therefore remains maximum even at high frequencies, which is no longer the case in solid metamaterials. Retaining this mobility is a major asset. This partly gelled capsule may form a chemical minireactor in which the nanoobjects may participate in new chemical reactions associating inorganicity with organicity.

The second step may be reversible. The benefit of the reversibility of this step is notably that, in the case of partly gelled capsules used as a chemical minireactor, it may be of interest to recover the reaction products by degelling the skin of the reactor in order to thereby recover the newly formed nanostructure. Thus, the first agglomerates may be destroyed, dismantled, by putting them into contact with chelating agents, chelators.

These chelating agents are specific chelating agents of the cations included in the structure of the agglomerates.

Thus, it is possible to select diethylene tetramine pentaacetic acid (DTPA) or ethylene diamine tetraacetic acid for Ca²⁺ cations, or trientine (triethylene tetramine, TETA) for the Fe³⁺ and Al³⁺ cations.

FIG. 3 is a photograph which shows the formation, in a test tube, of a first agglomerate or gelled agglomerate with the polysaccharide of FIG. 1, and carbon nanotubes as nanoobjects, and a calcium salt.

FIGS. 4A and 4B show an exemplary organization at a nanometric scale of a first agglomerate or gelled agglomerate comprising polysaccharides which are alginates and carbon nanotubes as a nanoobject, this agglomerate having been prepared from a second solution comprising a calcium salt.

In FIGS. 4A and 4B, each first agglomerate or gelled agglomerate comprises a single nanotube and a single polysaccharide.

In FIGS. 4A and 4B, it is noted that the cations act as crosslinking points (shaded area), i.e. in this case the calcium ions are put on the unoccupied sites —O⁻.

The first agglomerates, or gelled agglomerates, obtained at the end of the second step may be separated by any adequate separation method, for example by filtration. The first gelled agglomerates may be used as such in biological, microfluidic systems or as metamaterials for simulating the behavior of plasmas under electromagnetic radiation.

The gelled agglomerates such as spheres obtained during the second step may optionally in a third step be treated by impregnation for example with polyethylene glycol or any other water-soluble polymer or monomer, in solution (as an example for water, the optimum polyethylene glycol concentration is 20%). Examples of such polymers have already been given above.

These agglomerates either impregnated or not, being generally mixed with the (second) crosslinking aqueous solution, a separation step generally ensues, for example by filtration with a buchner, before the collected capsules are frozen for example by immersing them in liquid nitrogen. Instantaneous solidification minimizes salting-out (release) of the solvent, such as water, of the capsules maintaining maximum dispersion. This solidification, freezing, is in fact the first part of the freeze-drying treatment. The frozen capsules may optionally be stored in a freezer before proceeding with sublimation and with the subsequent treatments.

This solidification, freezing of the optionally impregnated agglomerates, is followed by a sublimation step which is the second part of the freeze-drying treatment. During this sublimation step, under the effect of the vacuum, the frozen solvent, such as ice, is removed, inside the capsules and optionally the polymer such as polyethylene glycol crystallizes.

The agglomerates may therefore be placed for example in an enclosure, chamber, cooled to −20° C. at the very least and under a high vacuum (10⁻³-10⁻⁷ mbar) in order to sublimate the frozen solvent such as ice and to optionally crystallize the polymer present such as polyethylene glycol.

Optionally, the freeze-drying treatment may comprise a third part during which the agglomerates are cold-dried.

It should be noted that this freeze-drying step may be accomplished even if the first solvent does not comprise any polymer or monomer and/or if the gelled agglomerates are not impregnated in a third step with a polymer or monomer, notably with a water-soluble polymer or monomer.

Freeze-drying may be achieved regardless of the solvent of the gelled agglomerates whether this is water or any other solvent or mixture of solvents. Generally, however, the solvent of the gelled agglomerates must contain water in majority.

At the end of the freeze-drying, there is substantially no longer any solvent in the freeze-dried agglomerates. The solvent content is generally less than 0.01% by mass.

If the solvent of the gelled agglomerates is composed of water, the water content of the freeze-dried agglomerates is generally less than 0.01% by mass.

The gelled agglomerates obtained at the end of the second step retain their shape and generally 90% of their volume after the freeze-drying.

The organization of the nanoobjects, such as CNTs, is retained in the freeze-dried capsules, as this is shown in FIG. 6.

Optionally, in order to remove at least partly the polysaccharide from the freeze-dried agglomerates, these freeze-dried agglomerates are subject to a heat treatment or an enzymatic treatment.

The heat treatment should generally be carried out at a sufficient temperature and for a sufficient time for removing at least partly the polysaccharide such as the alginate.

It may also be carried out at a temperature from 400 to 600° C., preferably from 500 to 550° C. for a duration from 1 to 5 hours, preferably from 1 to 3 hours, still preferably from 1 to 2 hours.

For example, a slow rise in temperature of 1° C./minute from room temperature up to 500° C., may be carried out, the temperature may be maintained at 500° C. for one hour and then lowered at a rate of 1° C./minute from 500° C. down to room temperature.

The conditions of the enzymatic treatment may easily be determined by one skilled in the art. Examples of these conditions have already been given above.

The gelled agglomerates, or the freeze-dried and optionally thermally or enzymatically treated agglomerates are then directly mixed through simple mechanical action to the granules of polymers or composites, i.e. mixtures of polymers and of inorganic fillers such as glass fibres, talc, mica particles, and particles other elements conventionally used in the field of the composite.

This mechanical action may comprise one or more operations. For example, only one extrusion may be carried out; or else simple mechanical mixing may be carried out, optionally followed by drying of the mixture, followed by extrusion of the mixture in an extruder.

The organization of the nanoobjects, such as CNTs, is retained after mixing of the capsules with a polymer such as PMMA (FIG. 7).

The invention will now be described with reference to the following examples, given as an illustration and not as a limitation:

### EXAMPLE 1

In this example the preparation of gelled agglomerates according to the invention (or gelled capsules) with 0.1% by mass containing carbon nanotubes, the freeze-drying of these gelled agglomerates, and the integration of these freeze-dried agglomerates into two polymers (polypropylene and polyamide 6) by a method according to the invention are described.

The preparation of the gelled agglomerates comprises the following successive steps:
In a beaker 1, 100 ml of deionized water with 0.5 g of alginate which is a sodium salt of alginic acid extracted from brown algae (“Alginic Acid sodium salt from brown algae”, CAS Number 9005-38-3, supplier Sigma Aldrich®) are poured in this order;
a magnetic stirrer is placed in the beaker 1 and the whole is mixed for two hours at 50° C.;
next, 0.1 g of multi-wall carbon nanotubes (MWCNT) (provided by Nanocyl®) purified to 95%, with an average diameter of 9.5 nm, and an average length of 1.5 μm, are added;
the nanotubes are dispersed under the action of ultrasound (in a Hielscher® 200S machine of 2006, frequency 24 kHz, with a microtip probe S7 with a diameter of 7 mm, adjustment of the amplitude to 30% ×175 μm i.e. 52 μm, i.e. an acoustic power density of 30%×300 W/cm², i.e. 90 W/cm²). The ultrasonic stirring time is two hours;
finally, 20 g of PEG 4000 (supplier VWR-Prolabo®) are added. By adding the PEG directly into the solution, dispersion, of the beaker 1, the impregnation step is thereby avoided;
in a beaker 2, 100 ml of deionized water and 1 g of CaCl₂ (CaCl₂ <<dried powder>> 97% purity CAS Number 10043-52-4) are poured and stirred with a magnetic stirrer at room temperature for one hour;
formation of the agglomerates, granules, is carried out automatically with a peristaltic pump adjusted to a flow rate of 0.8 ml/min. The endpiece, tip, used for forming the agglomerates is a Pasteur pipette positioned above a burette of 100 ml containing the solution of the beaker 2 (crosslinking solution). Drops of the contents of the beaker 1 which are found in the Pasteur pipette fall into the beaker 2;
the agglomerate, the capsule as illustrated in FIGS. 3 and 4 is formed instantaneously when the drop is detached by gravity from the Pasteur pipette, and falls into the contents of the burette. The capsule floats for a short time and sinks when the latter is completely crosslinked by the Ca²⁺ ions;
the agglomerates, capsules are then filtered in a “Buchner” containing a paper filter;
the agglomerates and the filter are then instantaneously immersed into liquid nitrogen in order to freeze the capsules;
the agglomerates may be stored in a freezer at −20° C. before being freeze-dried or more exactly subject to the <<sublimation>> part of the freeze-drying treatment;
freeze-drying is carried out in a commercial apparatus (LL1500 of Thermo-Fischer-Scientifique®) with a capacity of 1.5 kg/24 hours and with a maximum capacity of 3 kg. The temperature of the condenser is at −110° C.

The freeze-dried agglomerates prepared in this way are then mechanically mixed with 100 g of polypropylene granules.

The mixture is then dried at 40° C. for 12 hours before extrusion in a Thermo-Fisher Electron PRISM 16® extruder with 11 heating areas.

The screw profile has three shearing areas regularly distributed over a length of 1 m.

The temperature profile for the polypropylene is 170° C., 190° C., 200° C., 220° C., 230° C., 230° C., 230° C., 220° C., 200° C., 190° C., 180° C. The first value corresponds to the head of the extruder at the die and the last value corresponds to the area where the mixture of polymer granules and of the agglomerates is fed.

The freeze-dried agglomerates prepared as above were also introduced into polyamide 6.

The operating procedure is the same as the one already described above for polypropylene; only the temperature profile is changed, it is 250° C., 270° C., 270, 270° C., 270° C., 270° C., 270° C., 270° C., 270° C., 270° C., 250° C.

### EXAMPLE 2

In this example, preparation of gelled agglomerates according to the invention, containing nanotracers, freeze-drying of these gelled agglomerates, and integration of these freeze-dried agglomerates into two polymers (polypropylene and polyamide 6) by a method according to the invention, are described.

The preparation of the gelled agglomerates comprises the following successive steps:
In a beaker 1, 100 ml of deionized water with 0.5 g of alginate which is a sodium salt of the alginic acid extracted from brown algae (“Alginic Acid sodium salt from brown algae”, CAS Number 9005-38-3, supplier Sigma Aldrich®) are poured in this order;
a magnetic stirrer is placed in the beaker 1, and the whole is mixed for two hours at 50° C.;
next, 10 ml of an aqueous solution of nanotracers consisting of a rare earth oxide such as Gd₂O₃ doped with Europium at a 1% by mass concentration are added;
the nanotracers are dispersed under the action of ultrasound (Hielscher 2005® machine of 2006, frequency 24 kHz with a microtip probe S7 with a diameter of 7 mm, adjustment of the amplitude to 30%×175 μm i.e. 52 μm, i.e. an acoustic power density of 30%×300 W/cm, i.e. 90 W/cm²). The stirring time by the ultrasound is 10 minutes;
finally, 20 g of PEG 4000 (supplier VWR-Prolabo®) are added;
in a beaker 2, 100 ml of deionized water and 1 g of CaCl₂ (CaCl₂ <<dried powder>>, 97% purity, CAS Number 10043-52-4) are poured and stirred with a magnetic stirrer at room temperature for one hour;
the formation of the agglomerates, granules, aggregates, is carried out automatically with a peristaltic pump adjusted to a flow rate of 0.8 ml/min. The endpiece, nozzle, used for forming the agglomerates is a Pasteur pipette positioned above a 100 ml burette containing the solution of the beaker 2;
the agglomerate, the capsule containing the nanotracers is instantaneously formed when the drop is detached by gravity from the Pasteur pipette and falls into the contents of the burette. The capsule floats for a short instant and sinks when the latter is completely crosslinked by the Ca²⁺ ions;
the agglomerates are then filtered in a buchner containing a paper filter;
the agglomerates and the filter are then instantaneously immersed in liquid nitrogen in order to freeze the capsules;
the agglomerates may be stored in a freezer at −20° C. before being freeze-dried;
freeze drying is carried out in a commercial apparatus (LL1500 of Thermo-Fischer-Scientifique®) with a capacity of 1.5 kg/24 hours and with a maximum capacity of 3 kg. The temperature of the condenser is at −110° C.

The freeze-dried agglomerates prepared in this way are then mechanically mixed with 100 g of polypropylene granules.

The mixture is then dried at 40° C. for 12 hours before extrusion in a Thermo-Fisher Electron PRISM 16® extruder with 11 heating areas.

The screw profile has three shearing areas regularly distributed over a length of 1 m.

The temperature profile for the polypropylene is 170° C., 190° C., 200° C., 220° C., 230° C., 230° C., 230° C., 220° C., 200° C., 190° C., 180° C. The first value corresponds to the head of the extruder at the die, and the last value corresponds to the area where the mixture of polymer granules and of the agglomerates is fed.

The freeze-dried agglomerates prepared as above were also introduced into polyamide 6.

The operating procedure is the same as the one described above for polypropylene; only the temperature profile is changed, it is 250° C., 270° C., 270° C., 270° C., 270° C., 270° C., 270° C., 270° C., 270° C., 270° C., 250° C.

### EXAMPLE 3

In this example according to the invention, the preparation of gelled agglomerates (or gelled capsules) containing both carbon nanotubes and copper particles, the freeze-drying of these gelled agglomerates as well as the integration of these freeze-dried agglomerates in two polymers (polypropylene and polyamide 6) by a method according to the invention are described.

The preparation of the gelled agglomerates comprises the following successive steps:
In a beaker 1, 100 ml of deionized water with 0.5 g of alginate which is a sodium salt of the alginic acid extracted from brown algae (“Alginic Acid sodium salt from brown algae”, CAS Number 9005-38-3, supplier Sigma Aldrich®) are poured in this order;
a magnetic stirrer is placed in the beaker 1 and the whole is mixed for two hours at 50° C.;
next, 0.1 g of MWCNT (supplier Nanocyl®) 95% purified, average diameter 9.5 nm, average length 1.5 μm, are added;
the nanotubes are dispersed under the action of ultrasound (Hielscher 200S® machine of 2006, frequency 24 kHz, with a microtip probe S7 with a diameter of 7 mm, adjustment of the amplitude to 30%×175 μm, i.e. 52 μm, i.e. an acoustic power density of 30%×300 W/cm², i.e. 90 W/cm²). The stirring time by ultrasound is two hours;
next 0.1 g of copper particles are added which are also dispersed under the action of ultrasound (Hielscher 200S® machine of 2006, frequency 24 kHz, with a microtip probe S7 with a diameter of 7 mm, adjustment of the amplitude to 30%×175 μm, i.e. 52 μm, i.e. an acoustic power density of 30%×300 W/cm², i.e. 90 W/cm²). The stirring time by the ultrasound is 15 minutes;
finally, 20 g of PEG 4000 (supplier VWR-Prolabo®) are added;
in a beaker 2, 100 ml of deionized water and 1 g of CaCl₂ (CaCl₂ <<dried powder>>, purity 97%, CAS Number 10043-52-4) are poured and stirred with a magnetic stirrer at room temperature for one hour;
the formation of the agglomerates, granules is carried out automatically with a peristaltic pump adjusted to a flow rate of 0.8 ml/min. The endpiece, nozzle, used for forming the agglomerates is a Pasteur pipette positioned above a 100 ml burette containing the solution of the beaker 2;
the agglomerate, the capsule is instantaneously formed when the drop is detached by gravity from the Pasteur pipette and falls into the contents of the burette. The capsule floats for a short instant and sinks when the latter is completely crosslinked by Ca²⁺ ions.
the agglomerates, capsules are then filtered in a buchner containing a paper filter.

The agglomerates and the filter are then instantaneously immersed in liquid nitrogen in order to freeze the capsules.

The agglomerates may be stored in a freezer at −20° C. before being freeze-dried.

The freeze-drying is carried out in a commercial apparatus (LL1500 of Thermo-Fischer-Scientifique®) with a capacity of 1.5 kg/24 hours and with a maximum capacity of 3 kg. The temperature of the condenser is at −110° C.

The thereby prepared freeze-dried agglomerates are then mechanically mixed with 100 g of polypropylene granules.

The mixture is then dried at 40° C. for 12 hours before extrusion in a Thermo-Fisher Electron PRISM 16® extruder with 11 heating areas.

The screw profile has three shearing areas regularly distributed over a length of 1 m.

The temperature profile for the polypropylene is 170° C., 190° C., 200° C., 220° C., 230° C., 230° C., 230° C., 220° C., 200° C., 190° C., 180° C. The first value corresponds to the head of the extruder at the die and the last value corresponds to the area where the mixture of polymer granules and of the agglomerates is fed.

The freeze-dried agglomerates prepared as above were also introduced into polyamide 6.

The operating procedure is the same as the one already described above for polypropylene; only the temperature profile is changed, it is 250° C., 270° C., 270° C., 270° C., 270° C., 270° C., 270° C., 270° C., 270° C., 270° C., 250° C.

### REFERENCES

[1] TAKAYANAGI M., OGATA T., MORIKAWA M., KAI T., J. Macromol. Sci.-Phys., 1980, B17 (4), 591-615(1980).
[2] EP-A1-1 728 822.

1-42. (canceled)

43. An agglomerate or capsule prepared by freeze-drying of a first agglomerate or a first capsule, said first agglomerate or first capsule comprising a solvent, nanoobjects, or nanostructures coated with macromolecules of polysaccharides being distributed homogeneously in said first agglomerate or said first capsule, and said macromolecules forming, in at least one portion of the first agglomerate or the first capsule, a gel by crosslinking with positive ions.

44. The agglomerate according to claim 43, wherein the gel is formed in a totality of the first agglomerate.

45. The agglomerate according to claim 43,
wherein the gel is only formed at a surface of the first agglomerate, an inside of the first agglomerate being in a liquid state.

46. The agglomerate according to claim 43,
wherein a concentration of the nanoobjects or nanostructures is less than or equal to 5% by mass, of the total mass of the first agglomerate.

47. The agglomerate according to claim 43,
wherein the solvent comprises by volume 50% of water or more, preferably 70% of water or more, still preferably 99% of water or more, better 100% water.

48. The agglomerate according to claim 47,
wherein the solvent of the first agglomerate, when it does not comprise 100% water, further comprises at least one other solvent compound selected from among alcohols, aliphatic alcohols, ethanol, polar solvents, ketones, acetone, and their mixtures.

49. The agglomerate according to claim 43,
wherein the solvent of the first agglomerate further comprises a polymer or a monomer soluble therein.

50. The agglomerate according to claim 43,
wherein the nanoobjects are selected from among nanotubes, nanowires, nanoparticles, nanocrystals, and mixtures thereof.

51. The agglomerate according to claim 43, wherein the nanoobjects or nanostructures are chemically functionalized.

52. The agglomerate according to claim 43,
wherein material forming the nanoobjects or nanostructures is selected from among carbon, metals, metal alloys, metal oxides, doped rare earth oxides, organic polymers, and materials comprising several organic polymers.

53. The agglomerate according to claim 43,
wherein the nanoobjects are carbon nanotubes having single-walled or multi-walled carbon nanostructures, or nanoparticles of metals or metal alloys or metal oxides.

54. The agglomerate according to claim 43,
wherein the macromolecules of polysaccharide are selected from among pectins, alginates, alginic acid, and carrageenans.

55. The agglomerate according to claim 54,
wherein the alginates are extracted from brown algae Phaeophyceae, mainly Laminaria such as Laminaria hyperborea; and Macrocystis such as Macrocystis pyrifera.

56. The agglomerate according to claim 43, wherein the polysaccharide macromolecule has a molecular mass from 80,000 g/mol to 500,000 g/mol.

57. The agglomerate according to claim 43, wherein the first agglomerate is impregnated with at least one polymer or monomer soluble in the solvent of the first agglomerate, preferably with a water-soluble polymer or monomer such as polyethylene glycol (PEG).

58. The agglomerate according to claim 43, said agglomerate being further polymerized or crosslinked, or both.

59. The agglomerate according to claim 43,
wherein the first agglomerate after freeze-drying is further subject to a thermal or enzymatic treatment for removing at least partly the polysaccharide.

60. The agglomerate according to claim 59, the content of which in nanoobjects or nanostructures is from 50% to 100% by mass.

61. The use of the agglomerate according to claim 43 in microfluidic systems, or as a metamaterial for simulating behavior of plasmas under electromagnetic radiation.

62. A solid nanocomposite material with a polymer or composite matrix comprising the agglomerate according to claim 43, or the first agglomerate defined according to claims 43, wherein the nanoobjects or nanostructures are distributed homogeneously.

63. The nanocomposite material according to claim 62,
wherein one or more polymers of the matrix are selected from among aliphatic and apolar polymers comprising polyolefins, polyethylenes, polypropylenes, copolymers of cycloolefins, polystyrenes, polar polymers, polyamides, poly(meth)acrylates, PMMA, and mixtures thereof, polymers which melt or which are soluble in water; and
wherein the composite is selected from composite materials comprising at least one polymer and one inorganic filler.

64. A method for preparing the agglomerate according to claim 43, the method comprising the following steps:
a) dispersing nanoobjects or nanostructures in a first solvent comprising water in majority;
b) putting polysaccharide macromolecules and optionally a polymer or monomer soluble in the first solvent into solution into the first solvent, as a result of which a first solution is obtained;
c) preparing a third solution by putting the first solution in contact with a second solution in a second solvent comprising water in majority, of at least one water-soluble salt, capable of releasing in the second solution monovalent, divalent or trivalent cations, as a result of which a first agglomerate is obtained;
d) separating the first agglomerate from the third solution;
e) freeze-drying the first agglomerate; and
f) optionally, performing thermal or enzymatic treatment of the first freeze-dried agglomerate.

65. The method according to claim 64, wherein the first solvent comprises by volume 50% of water or more.

66. The method according to claim 65,
wherein the first solvent, when it does not comprise 100% of water, further comprises at least one other solvent compound selected from among alcohols, aliphatic alcohols, ethanol, polar solvents, ketones, acetone, and mixtures thereof.

67. The method according to claim 64, wherein the nanoobjects are selected from nanotubes, nanowires, nanoparticles, nanocrystals, and mixtures thereof.

68. The method according to claim 64,
wherein material forming the nanoobjects or nanostructures is selected from among carbon, metals, metals alloys, metal oxides, doped rare earth oxides, organic polymers, and materials comprising several organic polymers.

69. The method according to claim 64,
wherein the nanoobjects are carbon nanotubes having single-wall or multi-wall carbon nanostructures; or nanoparticles of metals or metals alloys or metal oxides.

70. The method according to claim 64, wherein the polysaccharide macromolecules are selected from among pectins, alginates, alginic acid and carrageenans.

71. The method according to claim 70, wherein the alginates are alginates extracted from brown algae and Macrocystis.

72. The method according to claim 64, wherein the macromolecules of polysaccharides have a molecular mass from 80,000 g/mol to 500,000 g/mol.

73. The method according to claim 64,
wherein the dispersion of the nanoobjects in the solvent and the putting of the polysaccharides into solution are two simultaneous operations, or two consecutive operations, the dispersion preceding the putting into solution, or vice versa.

74. The method according to claim 64, wherein a ratio of the number of macromolecules to the number of nanoobjects in the first solution is from 1 to 10, preferably equal to or close to 1.

75. The method according to claim 64,
wherein the content of nanoobjects and the content of macromolecules of polysaccharides are less than or equal to 5% by mass, of the mass of the first solvent.

76. The method according to claim 64, wherein the second solvent comprises by volume 50% of water or more.

77. The method according to claim 76,
wherein the second solvent, when it does not comprise 100% of water, further comprises at least one other solvent compound selected from among alcohols, aliphatic alcohols, ethanol, polar solvents, ketones, acetone, and mixtures thereof.

78. The method according to claim 64,
wherein divalent cations are selected from Cd²⁺, Cu²⁺, Ca²⁺, Co²⁺, Mn²⁺, Fe²⁺, Hg²⁺; monovalent cations are selected from Li⁺, Na⁺, K⁺, Rb⁺, Cs⁺, Ag⁺, Ti⁺, Au⁺; and trivalent cations are selected from Fe³⁺, and Al³⁺.

79. The method according to claim 64,
wherein the second solution comprises several salts so that a mixture of cations comprising at least one monovalent cation, at least one divalent cation, and at least one trivalent cation, are released in the second solution.

80. The method according to claim 64, further comprising:
a step of putting the agglomerate into contact with at least one chelating agent comprising diethylene tetramine pentaacetic acid (DTPA), ethylene diamine tetraacetic acid, or trientine (triethylene tetramine, TETA).

81. The method according to claim 64,
wherein at the end of step c) or d), the agglomerate is impregnated with a solution of a polymer or monomer soluble in the first solvent.

82. A method for preparing a nanocomposite material according to claim 62, further comprising:
incorporating at least one agglomerate according to claim 43, or of at least one first agglomerate as defined according to 43, in a polymer or composite matrix.

83. The method according to claim 82,
wherein the incorporation of the agglomerate in the polymer or composite matrix is carried out with a plastic engineering or processing method.

84. The method according to claim 82,
wherein the polymer of the matrix is selected from aliphatic and apolar polymers comprising polyolefins, polyethylenes, polypropylenes, copolymers of cycloolefins, polystyrenes, polar polymers, polyamides, poly(meth)acrylates, PMMA, and mixtures thereof, and polymers which melt or which are soluble in water; and
wherein the composite is selected from composite materials comprising a polymer and an inorganic filler.

85. The agglomerate according to claim 46,
wherein the concentration of the nanoobjects or nanostructures is less than or equal to 1% by mass, of the total mass of the first agglomerate.

86. The agglomerate according to claim 85,
wherein the concentration of the nanoobjects or nanostructures is from 10 ppm to 0.1% by mass, of the total mass of the first agglomerate.

87. The agglomerate according to claim 47, wherein the solvent comprises by volume 70% of water or more.

88. The agglomerate according to claim 87, wherein the solvent comprises by volume 99% of water or more.

89. The agglomerate according to claim 88, wherein the solvent comprises by volume 100% water.

90. The agglomerate according to claim 55, wherein the alginates are Laminaria or Laminaria hyperborea.

91. The agglomerate according to claim 55, wherein the Macrocystis are Macrocystis pyrifera.

92. The agglomerate according to claim 56, wherein the polysaccharide macromolecule has a molecular mass from 80,000 g/mol to 450,000 g/mol.

93. The agglomerate according to claim 57, wherein the at least one polymer or monomer soluble in the solvent of the first agglomerate is a water-soluble polymer or monomer.

94. The agglomerate according to claim 93, wherein the water-soluble polymer or monomer is polyethylene glycol (PEG).

95. The agglomerate according to claim 60, the content of which in nanoobjects or nanostructures is from 80% to 100% by mass.

96. The method according to claim 65, wherein the solvent comprises by volume 70% of water or more.

97. The method according to claim 96, wherein the solvent comprises by volume 99% of water or more.

98. The method according to claim 97, wherein the solvent comprises by volume 100% water.

99. The method according to claim 71, wherein the alginates are Laminaria or Laminaria hyperborea.

100. The method according to claim 71, wherein the Macrocystis are Macrocystis pyrifera.

101. The method according to claim 72, wherein the macromolecules of polysaccharides have a molecular mass from 80,000 g/mol to 450,000 g/mol.

102. The method according to claim 73,
wherein the dispersion of the nanoobjects in the solvent and the putting of the polysaccharides into solution are two consecutive operations.

103. The method according to claim 102,
wherein the dispersion of the nanoobjects in the solvent precedes the putting of the polysaccharides into solution.

104. The method according to claim 102,
wherein the dispersion of the nanoobjects in the solvent is performed after the putting of the polysaccharides into solution.

105. The method according to claim 74, wherein the ratio of the number of macromolecules to the number of nanoobjects in the first solution is about 1.

106. The method according to claim 75,
wherein the content of nanoobjects and the content of macromolecules of polysaccharides are less than or equal to 1% by mass, of the mass of the first solvent.

107. The method according to claim 106,
wherein the content of nanoobjects and the content of macromolecules of polysaccharides are from 10 ppm to 0.1% by mass, of the mass of the first solvent.

108. The method according to claim 76, wherein the second solvent comprises by volume 70% by volume of water or more.

109. The method according to claim 108, wherein the second solvent comprises by volume 99% by volume of water or more.

110. The method according to claim 109, wherein the second solvent comprises by volume 100% by volume of water.

111. The method according to claim 81,
wherein the solution of a polymer or monomer soluble in the first solvent is an aqueous solution of a water-soluble polymer or monomer.

112. The method according to claim 83,
wherein the incorporation of the agglomerate in the polymer or composite matrix is carried out with extrusion.
